(19) United States
(12) Patent Application Publication
(10) Pub. No.: US 2010/0069733 A1
(43) Pub. Date: Mar. 18, 2010

(54) ELECTROPHYSIOLOGY CATHETER WITH ELECTRODE LOOP

(76) Inventors: Nathan Kastelein, St. Louis, MO (US); Gareth T. Munger, St. Louis, MO (US)

Correspondence Address: HARNESS, DICKEY, & PIERCE, P.L.C, 7700 Bonhomme, Suite 400, ST. LOUIS, MO, 63105, US

(21) Appl. No.: 12/553,490

(22) Filed: Sep. 3, 2009

Related U.S. Application Data
(60) Provisional application No. 61/094,528, filed on Sep. 5, 2008.

(51) Int. Cl. A61B 5/042

(52) U.S. Cl. 600/374; 600/373

(57) ABSTRACT

A magnetically guidable electrophysiology catheter has an elongate catheter body having a proximal end and a distal end. At least one magnetically responsive element is disposed adjacent the distal end for aligning the distal end relative to an externally applied magnetic field. The portion of the catheter adjacent the distal end is formed in a generally planar loop, adjacent the distal end, with the distal end of the catheter projecting from the center of the loop, generally perpendicularly to the plane of the loop. A plurality of electrodes are disposed on the loop for measuring electrical activity in the tissue with which the loop is in contact.

### CROSS-REFERENCE TO RELATED APPLICATIONS

This application claims benefit of U.S. Provisional Patent Application Ser. No. 61/094,528, filed Sep. 5, 2008, the entire disclosure of which is incorporated by reference.

### BACKGROUND

This invention relates to electrophysiology catheters and in particular, to electrophysiology catheters that provide an electrode loop.

A wide variety of electrophysiology catheters have been developed to allow physicians to measure the electrical activity in the heart. One particular situation encountered, is measuring the electrical activity in the tissue surrounding an opening, for example, the pulmonary vein ostia in the left atrium, which is frequently the site of atrial fibrillation.

### SUMMARY

Embodiments of the present invention provide an electrophysiology catheter with an electrode loop that allows the measurement of electrical activity in the tissue with which it is in contact. Some embodiments of the present invention are particularly useful in measuring the electrical activity in the tissue surrounding an anatomical opening, such as the pulmonary vein ostia.

One preferred embodiment of the present invention provides a magnetically guidable electrophysiology catheter. Generally, this catheter comprises an elongate catheter body having a proximal end and a distal end. There is preferably at least one magnetically responsive element adjacent the distal end for aligning the distal end relative to an externally applied magnetic field. The portion of the catheter adjacent the distal end is formed in a generally planar loop. The distal end of the catheter projects from the center of the loop, generally perpendicularly to the plane of the loop. A plurality of electrodes are disposed on the loop for measuring electrical activity in the tissue with which the loop is in contact. In some embodiments the catheter body can include a generally helical portion formed in the catheter body proximal to the loop. In some embodiments the catheter body can include a portion intermediate the distal end and the loop extending from the loop to the center of the loop, substantially in the plane of the loop.

Still another preferred embodiment of the present invention provides a method of measuring the electrical activity in the tissue surrounding an anatomical opening. Generally, this method comprises orienting the distal end of a catheter with the anatomical opening. The catheter preferably has a loop formed therein proximal to the distal end, substantially in a plane generally perpendicular to the distal end of the catheter. The catheter is preferably oriented by applying a magnetic field to realign a magnetically responsive element associated with the distal end. The distal end of the catheter is advanced into the anatomical opening to bring the loop into contact with the tissue surrounding the opening. The electrical activity in the tissue in contact with the loop can be measured using at least one electrode on the loop.

Still another preferred embodiment of the present invention provides a method of magnetically navigating multiple magnetically navigable medical devices in a chamber. Generally, the method comprises orienting the distal end of a first medical device with an anatomical opening by applying a magnetic field sufficient to orient at least one magnetically responsive element associated with the distal end of the first medical device to cause the distal end of the first medical device to align with the anatomical opening. The distal end of the first medical device is then advanced into the anatomical opening, and held in place with the at least one magnetically responsive element suitably inserted into the anatomical opening. A magnetic field is applied to orient at least one magnetically responsive element associated with the distal end of a second medical device to cause the distal end of the second medical device to align relative to the magnetic field while the distal end of the first medical device is held in the anatomical opening. In some preferred embodiments, the distal end of the second medical device is aligned with an anatomical opening, and advanced into the anatomical opening and held in place with the at least one magnetically responsive element suitably inserted into the anatomical opening. The first medical device is released and a magnetic field is applied to orient at least one magnetically responsive element associated with the distal end of the first medical device to cause the distal end of the first medical device to align relative to the magnetic field.

Another preferred embodiment of this invention provides a magnetically guidable electrophysiology catheter comprising an elongate catheter body having a proximal end and a distal end. There is at least one magnetically responsive element proximal to the distal end for aligning the distal end relative to an externally applied magnetic field. The catheter body has a distal end portion with a generally planar loop formed therein, generally transverse to, and co-axial with the proximal portion of the catheter body. A plurality of electrodes are disposed on the loop for measuring electrical activity in the tissue with which the loop is in contact.

Still another preferred embodiment provides a method of measuring the electrical activity in the tissue surrounding an anatomical opening. Generally, the method comprises orienting the distal end of a catheter with the anatomical opening. The catheter has a loop formed therein in its distal end, substantially in a plane generally perpendicular to the proximal portion of the catheter. The catheter is aligned by applying a magnetic field to realign a magnetically responsive element associated with the proximal portion of the catheter: The proximal portion of the catheter is advanced into the anatomical opening to bring the loop into contact with the tissue surrounding the opening. The electrical activity in the tissue in contact with the loop can be measured using at least one electrode on the loop.

Embodiments of this invention provide devices for measuring the electrical activity on anatomical surfaces and in particular around anatomical openings

### BRIEF DESCRIPTION OF THE DRAWINGS

FIG. 1 is a perspective view of one preferred embodiment of a magnetically guidable electrophysiology catheter;

FIG. 2 is a side elevation view of the magnetically guidable electrophysiology catheter of FIG. 1, in a sheath;

FIG. 3 is an end elevation view of the magnetically guidable electrophysiology catheter of FIG. 1;

FIG. 3A is an enlarged longitudinal view of one possible construction of the distal end of the catheter;

FIG. 4 is a side elevation view of an alternate construction of the magnetically guidable electrophysiology catheter of FIG. 1;

FIG. 5 is a perspective view of a second preferred embodiment of a magnetically guidable electrophysiology catheter;

FIG. 6 is a perspective view of a third preferred embodiment of a magnetically guidable electrophysiology catheter;

FIG. 7 is perspective view of a fourth preferred embodiment of a magnetically guidable electrophysiology catheter, incorporating a force sensor proximal to the loop;

FIG. 8 is a perspective view of the distal end of an alternative construction of the electrophysiology catheter of the fourth embodiment, incorporating a plurality of individual force sensors;

FIG. 9 is a schematic view of a catheter with a loop that incorporates a set of fiber optic-based strain measurement arrays that can be used to estimate the shape of the catheter;

FIG. 10 is a schematic view of a catheter with a loop where the loop shape (such as diameter of the loop) can be mechanically controlled by incorporation of a suitable pull-wire-based mechanism;

FIG. 11 is a schematic depiction of a catheter with a loop where the loop shape (such as diameter of the loop) can be mechanically controlled by incorporation of a suitable pull-wire-based mechanism; and

FIG. 12 is a schematic depiction of a catheter with a loop where the loop shape (such as diameter of the loop) can be mechanically controlled by incorporation of a suitable pull-wire-based mechanism,

Corresponding reference numerals indicate corresponding parts throughout the several views of the drawings.

### DETAILED DESCRIPTION

A preferred embodiment of the present invention provides a magnetically guidable electrophysiology catheter. As shown in FIGS. 1 through 3, this magnetically guidable catheter 20 comprises an elongate catheter body having a proximal end 22 and a distal end 24. There is preferably at least one magnetically responsive element 26 adjacent the distal end 24 for aligning the distal end relative to an externally applied magnetic field. The magnetically responsive element can be made of any permanently magnetic or magnetically permeable material, but is preferably a neodymium-iron-born compound (Nd—Fe—B).

The portion of the catheter adjacent the distal end 24 is formed in a generally planar loop 28, adjacent the distal end. The distal end 24 of the catheter projects generally from the center of the loop 28, generally perpendicularly to the plane of the loop. A plurality of electrodes 30 are disposed on the loop 28 for measuring electrical activity in the tissue with which the loop is in contact. In a preferred embodiment, at least one of the electrodes 30 comprises magnetically responsive Platinum-Cobalt (PtCo) alloy. Such (magnetized) electrodes can help make the catheter touch the endocardial wall with good contact force. In a preferred embodiment, such PtCo electrodes are magnetized in a direction substantially parallel to the long axis of the catheter's distal end. When the distal end is inserted into an opening such as a pulmonary vein ostium, a magnetic field applied along a direction generally parallel to the axis of the vein near the opening, then causes the loop to firmly align with its normal generally parallel to the axis of the vein.

The catheter body can include a generally helical portion 32 formed in the catheter body proximal to the loop 28. The catheter body can also include a portion 34 intermediate the distal end and the loop 28, extending from the perimeter of the loop to the center of the loop, substantially in the plane of the loop. In some embodiments, the portion 34 can include a U-shaped bend (see 34′ in FIG. 4). This U-shaped bend allows the distal end 24 to protrude less from the plane of the loop 28, or even to be slightly proximal to the loop, to reduce how far the distal end penetrates the opening, and to reduce the possibility of trauma caused by the distal end 24.

As shown in FIG. 3A, the distal end 24 of the catheter 20 can be provided with a cap 36, which can overlie the magnetically responsive element 26, and a ring electrode 38 so that the distal end of the catheter can be used for electrical probing. The cap 36 and electrode 38 can be made of any suitable material such as platinum. Suitable lead wires extend to the proximal end 22 of the catheter 20, where they can be connected to suitable measuring equipment.

The catheter body preferably comprises a Nylon-based material, such as PEBAX® or some other flexible, resilient, biocompatible polymeric material that is capable of deforming but resiliently returns to its original configuration. The catheter can optionally include a shaping member (not shown), such as a core wire made from a shape memory alloy (e.g., nitinol) to facilitate the resilient shape retention of the distal end portion of the catheter. Instead of common straight/heat set wire sections, the stiffness profile of the core wire, and thus, of the distal end of the catheter 20 can be adjusted to improve performance by changing its diameter by centerless grinding or selective heat treating. Additionally, a composite wire structure could be employed. Specifically, the portions in the loop 28 and helical portion 32 can be spliced with straight linear elastic nitinol. Linear elastic nitinol has significantly better pushability characteristics while keeping bending stiffness the same. This would provide improved catheter navigation characteristics. The core structure could also be composed of a composite that includes braided or coiled nitinol.

The catheter preferably has a diameter of between about 1.5 mm and about 3.5 mm and preferably, between about 2 mm and about 3 mm. The loop 108 (in its reference or relaxed state) preferably has a diameter of between about 0.8 cm and about 5 cm and more preferably, between about 1 cm and about 3 cm. The distal end 104 preferably projects between about 0.5 cm and about 5 cm and more preferably, between about 1 cm and about 4 cm, from the loop.

The electrodes 30 on the loop can be monopolar or bi-polar, and can be made, for example, of Platinum-Iridium alloy. In a preferred embodiment, they can comprise magnetized PtCo alloy, as discussed earlier above. Lead wires (not shown) connected to electrodes 30 extend proximally through the catheter body to the proximal end where they can be connected to the appropriate equipment for capturing the electrical signals from the tissue in contact with the electrodes. There are preferably between about 2 and about 18 electrodes on the loop 28. They are preferably equally spaced around the loop 28, typically with between about 3 mm and about 25 mm between adjacent electrodes. The electrodes 30 are oriented on the loop 28 to face generally distally, so that they contact the surface of the tissue surrounding the opening into which the distal end 24 of the catheter is inserted. In one preferred embodiment, the electrodes 30 can be used to deliver Radio Frequency (RF) energy to the tissue the catheter loop contacts. In such a case, the catheter is both a therapeutic and diagnostic device, the RF energy serving to electrically isolate endocardial regions by locally destroying abnormal electrical pathways.

Another preferred embodiment of the present invention provides a method of measuring the electrical activity in the tissue surrounding an anatomical opening, such as a pulmonary vein ostium. Generally, this method comprises orienting the distal end 24 of the catheter 20 with the anatomical opening. The catheter 20 has a loop 28 formed therein proximal to the distal end 24. The loop 28 is substantially in a single plane generally perpendicular to the distal end of the catheter 20. The catheter 20 is preferably oriented by applying a magnetic field, for example from a magnetic navigation system available from Stereotaxis, Inc., St. Louis, Mo., of a direction and strength sufficient to realign the magnetically responsive element 26 associated with the distal end 24. The distal end 24 of the catheter is advanced into the pulmonary vein ostium or anatomical opening to bring the loop 28 into contact with the tissue surrounding the opening. The electrical activity in the tissue in contact with the loop 28 can be measured using at least one electrode on the loop.

In some embodiments, the catheter 20 is constructed so that when the distal end 24 of the catheter 20 is advanced into the opening, the helical portion 32 can resiliently collapse. This collapse, which could be viewed on x-ray imaging or measured, for example, by strain gauges or optically, helps ensure that the electrodes 30 on the loop 28 contact the tissue with sufficient force to obtain accurate measurement of the electrical activity. The resiliency of the materials from which the catheter 20, and in particular, the helical portion 32 is made moderates the contact force, reducing the risk of damage to the tissue. The helical portion 32 stores axial spring force that can be used to hold the loop against the wall when advanced manually or manipulated remotely by an advancer device. The helical shape absorbs movement and maintains most of the push force primarily in an axial direction in the presence of heart pulsation. While the helical portion 32 is preferred, some other configuration could be used. For example, if a straight shaft were used, push force would be stored as shaft buckling, however this would result in some deflecting of the position of the loop 28, altering electrode contact forces, and deflecting the distal end 24.

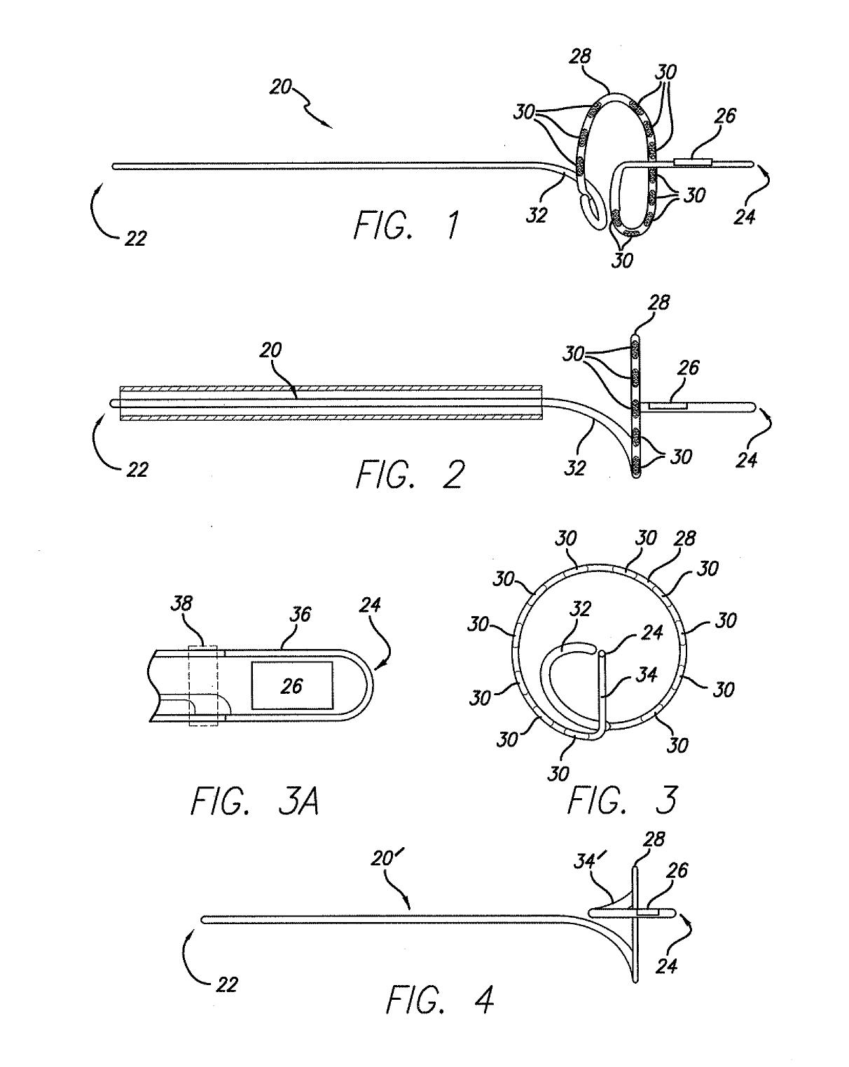

In at least some embodiments, the catheter 20 can be deployed from a sheath 40 (FIG. 2). In addition to protecting and facilitating the introduction of the catheter 20, the sheath can provide useful torquing of the distal end 24. Relative axial movement of the catheter 20 (and in particular the helical portion 32) and the sheath 40 will cause some turning off the distal end of the catheter 20. This causes the electrodes 30 to reposition (rotate) to provide additional information about the electrical activity in the area.

Still another preferred embodiment of the present invention provides a method of magnetically navigating multiple magnetically navigable medical devices in an anatomical volume or chamber, such as a chamber of the heart. Generally, the method comprises orienting the distal end of a first medical device, such as catheter 20 with a first anatomical opening. For example the distal end 24 could be disposed in the left atrium of the heart, and oriented with a pulmonary vein ostium. The catheter 20 is preferably oriented by applying a magnetic field, for example, from a magnetic navigation system available from Stereotaxis, Inc., St. Louis, Mo., of a direction and strength sufficient to orient at least one magnetically responsive element 26 associated with the distal end 24 of the first medical device 20, to cause the distal end of the first medical device to align with the anatomical opening. The distal end 24 of the first medical device 20 is then advanced into the anatomical opening, and held in place with the at least one magnetically responsive element 26 in the anatomical opening. The pressure retains the at least one magnetically responsive element 26 in the first anatomical opening.

A magnetic field is applied to orient at least one magnetically responsive element 26′ associated with the distal end 24′ of a second medical device 20′, to cause the distal end of the second medical device to align relative to the magnetic field while the distal end 24 of the first medical device 20 is held in the anatomical opening. The distal end 24′ of the second medical device 20′ could be aligned with a second anatomical opening in the same chamber, and advanced into the anatomical opening and held in place with the at least one magnetically responsive element 26′ in the first anatomical opening. The first medical device 20 is released and a magnetic field is applied to orient the at least one magnetically responsive element 26 associated with the distal end 24 of the first medical device 20, to cause the distal end of the first medical device to align relative to the magnetic field.

Inserting the distal end 24 into the opening is visible on x-ray or fluoroscope imaging, allowing the physician to confirm placement of the device, and ensuring that the electrodes are reliably centered around the opening. This also facilitates automation. While navigating a second device, such as device 20′ or an ablation catheter, the distal end 24 may “wiggle” somewhat, but the spring force provided by the helical portion 32 will help hold the loop 28 in position. In instances where the second device is an ablation catheter, it will generally be pointed in the same direction as the opening in which the distal end 24 of the catheter 20 is disposed, so this “wiggle” will be minor and will not effect the position of loop 28.

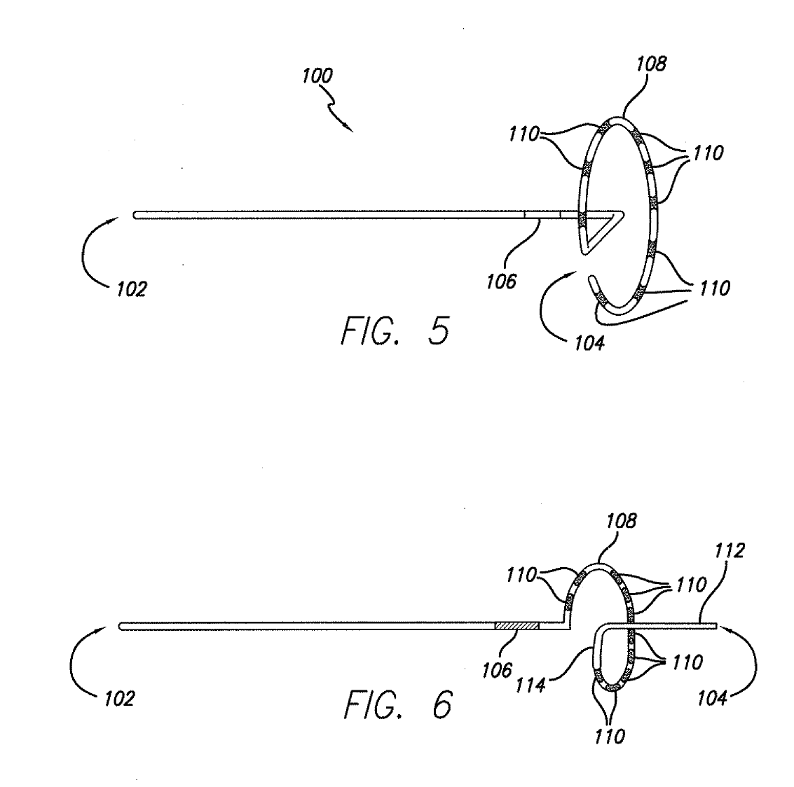

A second preferred embodiment of this invention provides a magnetically guidable electrophysiology catheter. As shown in FIG. 5, this magnetically guidable catheter 100 comprises an elongate catheter body having a proximal end 102 and a distal end 104. There is at least one magnetically responsive element 106 proximal to the distal end 104, for aligning the distal end of the catheter 100 relative to an externally applied magnetic field. The catheter 100 is preferably oriented by applying a magnetic field, for example, from a magnetic navigation system available from Stereotaxis, Inc., St. Louis, Mo., of a direction and strength sufficient to realign the magnetically responsive element 106 adjacent to the distal end 104. The portion of the catheter 100 distal to the at least one magnetically responsive element 106 is formed in a generally planar loop 108, adjacent the distal end 104, with the proximal portion of the catheter 100, with the magnetically responsive elements 106, projecting from the center of the loop 108, generally perpendicularly to the plane of the loop. A plurality of electrodes 110 are disposed on the loop for measuring electrical activity in the tissue with which the loop is in contact.

A third preferred embodiment of an electrophysiology catheter, indicated as 100′ in FIG. 6, is similar in construction to the second preferred embodiment shown in FIG. 5, and corresponding parts are identified with corresponding reference numerals. However, catheter 100′ includes a distally extending nose portion 112, connected to the end of the loop 108, by a radially extending segment 114, generally in the plane of the loop. The nose 112 allows the distal end of the catheter to be anchored in an anatomical opening, such as the ostia of a blood vessel.

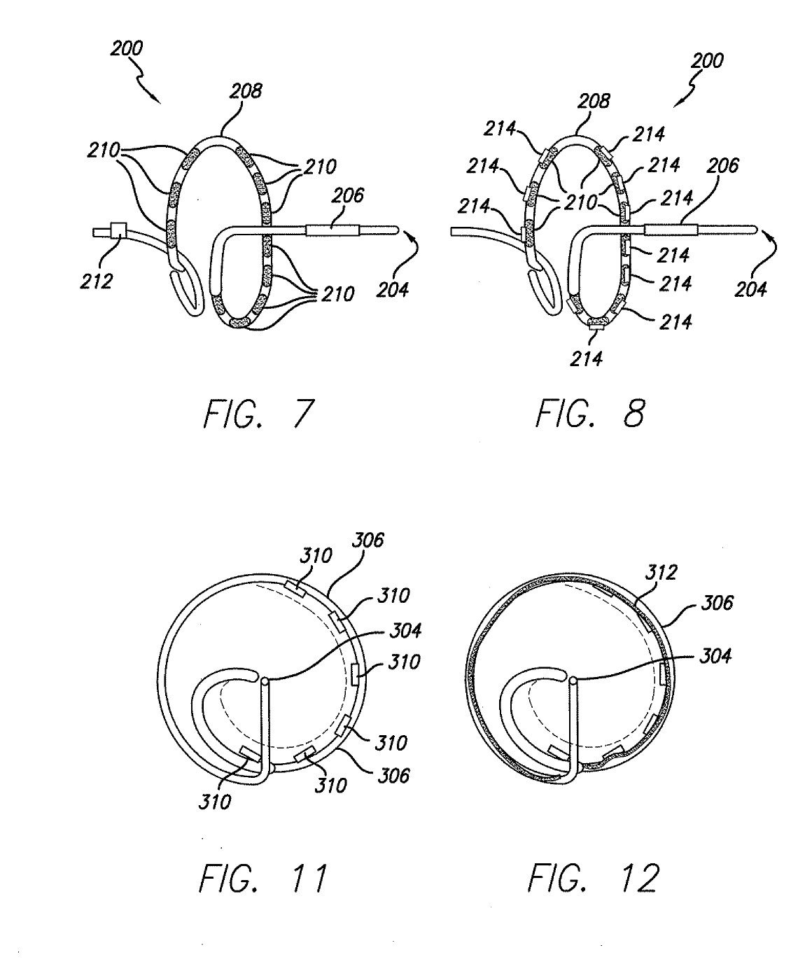

FIGS. 7 and 8, show embodiments of catheters with loops that incorporate at least one force sensing element. In one preferred embodiment, the distal end portion of an electrophysiology catheter 200 has a magnetically responsive element 206 proximal to the distal end 204. A generally planar loop 208 is formed proximal to the distal portion. A plurality of electrodes 210 are disposed on the loop 208. As shown in FIG. 7, a force sensor 212 is disposed proximal to the loop 208, for measuring the force applied by/to the loop. This axial force relates to the force applied between the electrodes 210, on the loop 208, and the adjacent tissue. As shown in FIG. 8, at least one force sensor 214 is associated with each electrode 210, for directly measuring the force applied by/to the loop in the vicinity of each electrode. The force sensors can be based on Micro-Electro-Mechanical-Systems (MEMS) technology, with leads attached to it for purposes of conveying an electrical signal to a force-sensing/signal processing system, connected to the proximal end of the catheter.

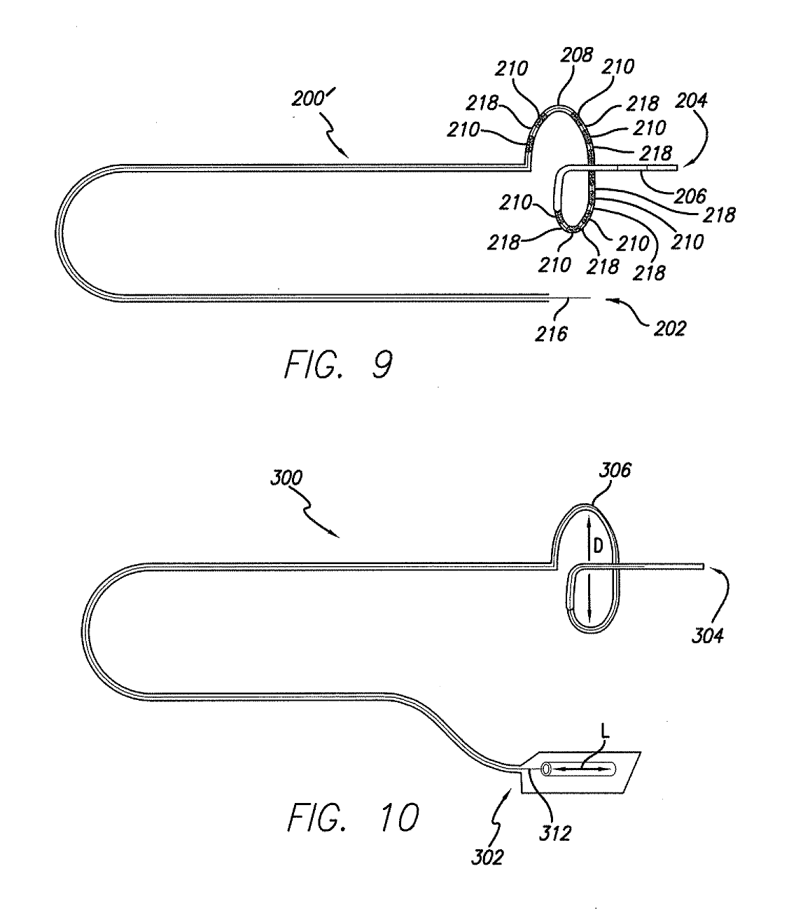

In an alternate embodiment, indicated as 200″ in FIG. 9, the force sensor can be in the form of a set of fiber-optic based strain measuring elements which measure local strain in different regions of the distal portion of the catheter. The catheter 200″ is similar to catheters 200, and corresponding parts are identified with corresponding reference numerals. The catheter 200″ incorporates at least one optical fiber 216 for conveying light to and from strain sensors in the form of Bragg arrays 218 (from which strain can be measured by light interference measurements; such arrays are commercially available, for example, from Luna Technologies Inc.). An optical system connected to the proximal end of the catheter, incorporates a signal processing system for conversion of strain measurements to catheter shape/loop geometry determination. Further data/signal processing can be used to determine local force estimates from such shape measurements, for example, based on a mathematical model of the distal end of the catheter 200″ or from a look up table.

FIGS. 10-12, show a preferred embodiment 300 of the looped catheter of the present invention, where the catheter incorporates a pull-wire system for mechanically controlled changes of loop geometry (such as loop diameter). The catheter 300 has a proximal end 302 and a distal end 304. There is a generally planar loop 306 formed in the catheter proximal to the distal end 304. The loop 306 generally extends in a plane perpendicular to the orientation of the portions of the catheter proximal to and distal to the loop. A magnetic responsive element can be disposed in the catheter, preferably proximal to or distal to the loop 306, so that the distal end 304 of the catheter can be magnetically navigated. A pull-wire 310 extends through the proximal portion of the catheter 300, and its distal end is preferably anchored to the catheter on the distal side of the loop 306, as shown in FIG. 10. As described above, a plurality of electrodes 310 can be disposed on the loop 306 for measuring electrical signals and/or ablating tissue. Pulling or pushing the pull-wire 312 at the proximal end of the catheter causes the diameter D of the loop 306 to increase or decrease. This allows the user to selectively change the size of the loop over which the electrodes on the loop are applied. The proximal end of the wire can be attached to a pull-wire manipulation mechanism that is either controlled by a knob or lever operated directly by a physician or is remotely manipulated by computer control of a driver mechanism. In one preferred embodiment, the proximal shaft of the catheter passes through a catheter advancement/rotation mechanism that can be remotely controlled from a computerized system to advance/retract or rotate the catheter.

As shown in FIGS. 11 and 12, the loop 306 positions the electrodes 310 in a ring of a first diameter, but when the pull-wire 312 is tensioned, the size of the ring is compressed (represented by the dashed lines in FIG. 11). FIG. 12, shows the pull-wire 312 when it is not under tension.

The catheter body preferably comprises a Nylon-based material, such as PEBAX® or some other flexible, resilient, biocompatible polymeric material that is capable of deforming, but resiliently returns to its original configuration. The catheter 100 can optionally include a shaping member (not shown) made from a shape memory alloy (e.g., nitinol) to facilitate the resilient shape retention of the distal end portion of the catheter.

The catheter preferably has a diameter of between about 1.5 mm and about 3.5 mm and preferably, between about 2 mm and about 3 mm. The loop 108 (in its reference or relaxed state) preferably has a diameter of between about 0.8 cm and about 5 cm and more preferably, between about 1 cm and about 3 mm. The distal end 104 preferably projects between about 0.5 cm and about 5 cm and more preferably, between about 1 cm and about 4 cm, from the loop.

The electrodes 110 on the loop can be monopolar or bi-polar, and can be made, for example, of Platinum-Iridum alloy. In a preferred embodiment, they can comprise magnetized PtCo alloy, as discussed earlier above. Lead wires (not shown) connected to electrodes 110, extend proximally through the catheter body to the proximal end, where they can be connected to the appropriate equipment for capturing the electrical signals from the tissue in contact with the electrodes. There are preferably between about 2 and about 18 electrodes on the loop 108. They are preferably equally spaced around the loop 108, typically with between about 3 mm and about 25 mm between adjacent electrodes. The electrodes 110 are oriented on the loop 108 to face generally distally so that they contact the surface of the tissue surrounding the opening into which the distal end 104 of the catheter is inserted.

Still another preferred embodiment provides a method of measuring the electrical activity in the tissue surrounding an anatomical opening. Generally, the method comprises orienting the distal end of a catheter 100 with an anatomical opening or other point of interest on the surface. The catheter 100 has a loop 108 formed therein in its distal end, substantially in a plane generally perpendicular to the proximal portion of the catheter. The catheter 100 is aligned by applying a magnetic field to realign a magnetically responsive element 106 associated with the proximal portion of the catheter. The proximal portion of the catheter is advanced toward thus surface of the tissue to bring the loop 108 into contact with the tissue surrounding the opening. The electrical activity in the tissue in contact with the loop 108 can be measured using at least one electrode 110 on the loop.

When used to measure the electrical activity around an opening, such as a pulmonary vein ostia, the portion of the catheter 100 proximal to the loop 108 (where the at least one magnetically responsive element 106 is located) can be aligned with and advanced at least partially into the opening. This helps ensure that the electrodes 110 on the loop 108 contact the tissue with sufficient force to obtain accurate measurement of the electrical activity. The resiliency of the materials from which the catheter 100 is made moderates the contact force, reducing the risk of damage to the tissue.

In one preferred embodiment, the catheter can also incorporate at least one electromagnetic location sensing element that can be used to localize the catheter within the anatomy with good accuracy. Such a location sensor for example, receives electromagnetic signals emitted by a set of transmitters that are part of a localization system; the location and orientation of the catheter in space relative to the transmitters can be determined from analysis of the signals received by the location sensor. Preferably, two location sensors are incorporated, with one sensor placed at the distal tip of the catheter, and one sensor placed either just proximal to the catheter loop or at the proximal portion of the catheter loop. When two location sensors are incorporated, the location and orientation information obtained from the two sensors can be used to estimate the shape of the distal portion of the catheter (including the loop itself), from which forces acting on the distal portion of the catheter can be estimated. When the catheter is equipped with at least one such location sensor, the catheter distal tip can be advanced/retracted by a catheter advancement/rotation mechanism and deflected by magnetic steering, so that it can be guided to a desired location, such as a pulmonary vein ostium under closed-loop or open-loop feedback control.

What is claimed is:

1. A magnetically guidable electrophysiology catheter comprising an elongate catheter body having a proximal end and a distal end; at least one magnetically responsive element adjacent the distal end for aligning the distal end relative to an externally applied magnetic field; the portion of the catheter adjacent the distal end being formed in a generally planar loop, adjacent the distal end, with the distal end of the catheter projecting from the center of the loop, generally perpendicularly to the plane of the loop; and a plurality of electrodes on the loop for measuring electrical activity in the tissue with which the loop is in contact.

2. The magnetically guidable electrophysiology catheter according to claim 1 wherein the catheter body includes a generally helical portion formed in the catheter body proximal to the loop.

3. The magnetically guidable electrophysiology catheter according to claim 1 wherein the catheter body includes a portion intermediate the distal end and the loop extending from the loop to the center of the loop, substantially in the plane of the loop.

4. A magnetically guidable electrophysiology catheter comprising an elongate catheter body having a proximal end and a distal end; at least one magnetically responsive element adjacent the distal end for aligning the distal end relative to an externally applied magnetic field; the catheter body having a distal end portion with a generally planar loop formed therein, a helical transition portion between the proximal portion and the catheter body loop, so that the loop is generally transverse to, and co-axial with the proximal portion of the catheter body, a generally radially extending portion extending from the loop to the center of the loop, the distal end of the catheter projecting from the center of the loop, generally perpendicularly to the plane of the loop; and a plurality of electrodes on the loop for measuring electrical activity in the tissue with which the loop is in contact.

5. The catheter of claim 4, wherein the electrode material comprises magnetized Platinum-Cobalt alloy.

6. The catheter of claim 4, wherein the catheter further incorporates a force sensor in the form of a MEMS-based force sensing element proximal to the loop in the distal end portion.

7. The catheter of claim 4, wherein the catheter further incorporates at least one optical fiber and at least one set of optical strain sensing elements in the form of a Bragg array.

8. The catheter of claim 4, wherein the electrodes are capable of delivery of Radio-Frequency energy to tissue contacted by the loop in the distal end portion.

9. The catheter of claim 4, wherein the distal portion of the catheter incorporates at least one location sensor for sensing location within a patient's anatomy.

10. The catheter of claim 4, wherein the catheter further incorporates a pull-wire for actuation of the shape of the loop in the catheter.

11. A magnetically guidable electrophysiology catheter comprising an elongate catheter body having a proximal end and a distal end; at least one magnetically responsive element adjacent the distal end for aligning the distal end relative to an externally applied magnetic field; the portion of the catheter proximal to the at least one magnet being configured to have a loop therein, substantially in a plane perpendicular to the portions of the catheter body proximal and distal to the loop; and a plurality of electrodes on the loop for measuring electrical activity in the tissue with which the loop is in contact.

12. The catheter of claim 11, wherein the distal portion of the catheter incorporates at least one location sensor for sensing location within a patient's anatomy.

13. The catheter of claim 11, wherein the catheter further incorporates a pull-wire for actuation of the shape of the loop in the catheter.

14. A method of measuring the electrical activity in the tissue surrounding an anatomical opening, the method comprising orienting the distal end of a catheter with the anatomical opening, the catheter having a loop formed therein proximal to the distal end, substantially in a plane generally perpendicular to the distal end of the catheter, by applying a magnetic field to realign a magnetically responsive element associated with the distal end; and advancing the distal end of the catheter into the anatomical opening to bring the loop into contact with the tissue surrounding the opening; and measuring electrical activity in the tissue in contact with the loop using at least one electrode on the loop.

15. The method of claim 14, wherein the catheter is advanced by remote operation of a catheter advancement mechanism.

16. A method of measuring the electrical activity in the tissue surrounding an anatomical opening, the method comprising orienting the distal end of a catheter with the anatomical opening by applying a magnetic field sufficient to orient at least one magnetically responsive element associated with the distal end of the catheter to cause the distal end of the catheter to align with the anatomical opening; advancing the distal end of the catheter into the anatomical opening to bring a generally transversely extending planer loop formed in the catheter adjacent the distal end, into contact with the tissue surrounding the opening, and measuring the electrical activity in the tissue in contact with the loop using at least one electrode carried on the loop.

17. A method of magnetically navigating multiple magnetically navigable medical devices in a chamber, the method comprising: orienting the distal end of a first medical device with an anatomical opening by applying a magnetic field sufficient to orient at least one magnetically responsive element associated with the distal end of the first medical device to cause the distal end of the first medical device to align with the anatomical opening; advancing the distal end of the first medical device into the anatomical opening, and holding the first medical device in place with the at least one magnetically responsive element in the anatomical opening; applying a magnetic field to orient at least one magnetically responsive element associated with the distal end of a second medical device to cause the distal end of the second medical device to align relative to the magnetic field while the distal end of the first medical device is held in the anatomical opening.

18. The method according to claim 8 wherein the distal end of the second medical device is aligned with an anatomical opening, and further comprising advancing the distal end of the second medical device into the anatomical opening, holding the second medical device in place with the at least one magnetically responsive element in the anatomical opening; releasing the first medical device, and applying a magnetic field to orient at least one magnetically responsive element associated with the distal end of the first medical device to cause the distal end of the first medical device to align relative to the magnetic field.

19. A magnetically guidable electrophysiology catheter comprising an elongate catheter body having a proximal end and a distal end; at least one magnetically responsive element proximal to the distal end for aligning the distal end relative to an externally applied magnetic field; the portion of the catheter distal to the at least one magnetically responsive element being formed in a generally planar loop, adjacent the distal end, with the proximal portion of the catheter with the magnetically responsive elements catheter projecting from the center of the loop, generally perpendicularly to the plane of the loop; and a plurality of electrodes on the loop for measuring electrical activity in the tissue with which the loop is in contact.

20. A magnetically guidable electrophysiology catheter comprising an elongate catheter body having a proximal end and a distal end; at least one magnetically responsive element proximal to the distal end for aligning the distal end relative to an externally applied magnetic field; the catheter body having a distal end portion with a generally planar loop formed therein, generally transverse to, and co-axial with the proximal portion of the catheter body; and a plurality of electrodes on the loop for measuring electrical activity in the tissue with which the loop is in contact.

21. A magnetically guidable electrophysiology catheter comprising an elongate catheter body having a proximal end and a distal end; at least one magnetically responsive element adjacent the distal end for aligning the distal end relative to an externally applied magnetic field; the portion of the catheter distal to the at least one magnetically responsive element being configured to have a loop therein, substantially in a plane perpendicular to the portions of the catheter body proximal and distal to the loop; and a plurality of electrodes on the loop for measuring electrical activity in the tissue with which the loop is in contact.

22. A method of measuring the electrical activity in the tissue surrounding an anatomical opening, the method comprising orienting the distal end of a catheter with the anatomical opening, the catheter having a loop formed therein in its distal end, substantially in a plane generally perpendicular to the proximal portion of the catheter, by applying a magnetic field to realign a magnetically responsive element associated with the proximal portion of the catheter; and advancing the proximal portion of the catheter into the anatomical opening to bring the loop into contact with the tissue surrounding the opening; and measuring electrical activity in the tissue in contact with the loop using at least one electrode on the loop.

23. A method of measuring the electrical activity in the tissue surrounding an anatomical opening, the method comprising orienting the distal end of the catheter with the anatomical opening by applying a magnetic field sufficient to orient at least one magnetically responsive element associated with the catheter to cause the distal end of the catheter to align with the anatomical opening; advancing the distal end of the catheter into the anatomical opening to bring a generally transversely extending planer loop formed in the catheter distal to the at least one magnetically responsive element, into contact with the tissue surrounding the opening, and measuring the electrical activity in the tissue in contact with the loop using at least one electrode carried on the loop.
